(19) United States
(12) Patent Application Publication
(10) Pub. No.: US 2013/0144143 A1
(43) Pub. Date: Jun. 6, 2013

(54) PROBE STRUCTURE CAPABLE OF MEASURING pH LEVEL

(71) Applicants: Jin Seok KIM, Seoul (KR); Jun-Kyo Francis SUH, Seoul (KR)

(72) Inventors: Jin Seok KIM, Seoul (KR); Jun-Kyo Francis SUH, Seoul (KR)

(21) Appl. No.: 13/684,793

(22) Filed: Nov. 26, 2012

(30) Foreign Application Priority Data
Dec. 5, 2011 (KR) 10-2011-0128922

(51) Int. Cl. A61B 5/145

(52) U.S. Cl. CPC A61B 5/14503
USPC 600/361

(57) ABSTRACT

The probe structure capable of measuring a pH level according to an embodiment of the present disclosure includes: a probe unit inserted into an experiment target; a fixing body connected to a terminal of the probe unit and fixing the probe unit; an electrode array disposed at a front end of the probe unit and sensing a neural signal from the experiment target and a pH level; a reference electrode disposed at the front end of the probe unit to be spaced apart from the electrode array by a predetermined interval and sensing a reference signal for pH level measurement; an electric wire electrically connected to the electrode array and the reference electrode; and a measured signal collecting electrode integrated at the fixing body and collecting measured signals transmitted through the electric wire.

### CROSS-REFERENCE TO RELATED APPLICATION

This application claims priority to Korean Patent Application No. 10-2011-0128922, filed on Dec. 5, 2011, and all the benefits accruing therefrom under 35 U.S.C. §119, the contents of which in its entirety are herein incorporated by reference.

### BACKGROUND

1. Field

The present disclosure relates to a probe structure capable of measuring a pH level, and more particularly, to a probe structure capable of measuring a pH level as well as obtaining a neural signal as conventionally by adding a reference electrode to the probe structure.

2. Description of the Related Art

Recently, a study for treating brain diseases and establishing brain activities by stimulating cranial nerves of an experiment target and then sensing and analyzing resultant signals has been actively researched.

In order to directly stimulate cranial nerves of an experiment target and collect resultant information, a nerve probe capable of being inserted into the experiment target is used. In addition, in order to detect information according to cranial nerve stimulation as much as possible, a subminiature nerve probe at which an electrode array is integrated has been developed.

Conventional nerve probes generally apply an electric stimulation to cranial nerves by using electrodes integrated at a probe unit in order to stimulate the cranial nerves. If an electric stimulation is applied to the cranial nerves as described above, the cranial nerves may be damaged. In addition, since substances of the brain are electrically conductive, it is impossible to apply a local stimulation to a desired portion.

Therefore, a method of applying an optical stimulation to cranial nerves by using light and then collecting resultant neural signals has recently been introduced.

Meanwhile, since conventional nerve probes generally places emphasis on obtaining neural signals of cranial nerves, the nerve probes leave much room for improvement by adding functions other than obtaining neural signals.

### RELATED LITERATURES

### Patent Literature

Literature 1: Korean Patent Registration No. 10-0404783 (Research Institute of Industrial Science and Technology), Oct, 28, 2003, Abstract, claim 1, FIG. 3

Literature 2: Korean Patent Registration No. 10-0441664 (Research Institute of Industrial Science and Technology), Jul. 15, 2004, Abstract, claim 1, FIG. 3

### SUMMARY

The present disclosure is directed to providing a probe structure capable of measuring a pH level as well as obtaining a neural signal as conventionally with a simple configuration where a reference electrode is added to the probe structure.

In one aspect, there is provided a probe structure capable of measuring a pH level, which includes: a probe unit inserted into an experiment target; a fixing body connected to a terminal of the probe unit and fixing the probe unit; an electrode array disposed at a front end of the probe unit and sensing a neural signal from the experiment target and a pH level; a reference electrode disposed at the front end of the probe unit to be spaced apart from the electrode array by a predetermined interval and sensing a reference signal for pH level measurement; an electric wire electrically connected to the electrode array and the reference electrode; and a measured signal collecting electrode integrated at the fixing body and collecting measured signals transmitted through the electric wire.

The electrode array may include: a working electrode for applying an electric stimulation to the experiment target; and a neural signal recording electrode for obtaining a neural signal of the experiment target changed by the working electrode.

The probe structure may further include an optical fiber for transmitting an optical signal to the probe unit.

The probe unit may be made of glass which allows optical signal transmission.

The probe unit may be made of polymer which allows optical signal transmission.

The probe structure may further include an optical waveguide connected to the optical fiber and fixed and attached to the surface of the probe unit along the longitudinal direction of the probe unit.

A groove may be formed in the probe unit along the longitudinal direction to accommodate the optical waveguide.

The reference electrode may be made of a nanoporous Au electrode to minimize the change of charge capacity.

The probe structure of the present disclosure may measure the change of a pH level as well as obtain a neural signal as conventionally without changing size and thickness just with a simple configuration where a reference electrode is added to the probe structure.

### BRIEF DESCRIPTION OF THE DRAWINGS

The above and other aspects, features and advantages of the disclosed exemplary embodiments will be more apparent from the following detailed description taken in conjunction with the accompanying drawings in which:

FIG. 1 is a perspective view showing a probe structure capable of measuring a pH level according to an embodiment of the present disclosure;

FIG. 2 is an enlarged view showing the A portion of FIG. 1;

FIG. 3 is a perspective view showing a probe structure capable of measuring a pH level according to another embodiment of the present disclosure; and

FIG. 4 is an enlarged view showing the B portion of FIG. 3.

### DETAILED DESCRIPTION OF MAIN ELEMENTS

10, 100: probe structure

110: probe unit

112: groove

120: fixing body

130: optical fiber

140: electrode array

150: reference electrode

160: electric wire

170a, 170b: measured signal collecting electrode

180: optical waveguide

### DETAILED DESCRIPTION

Hereinafter, a probe structure capable of measuring a pH level according to an embodiment of the present disclosure will be described in detail with reference to the accompanying drawings.

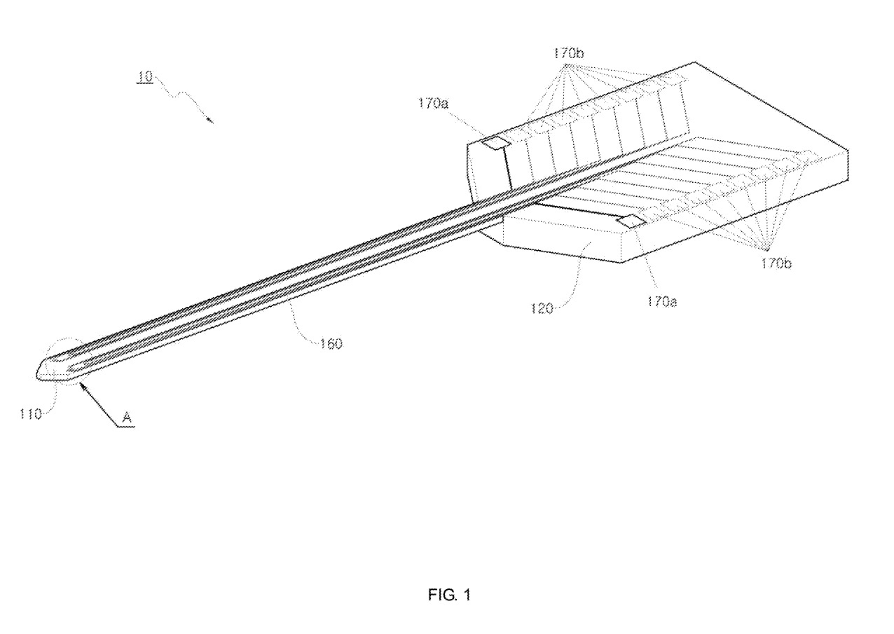

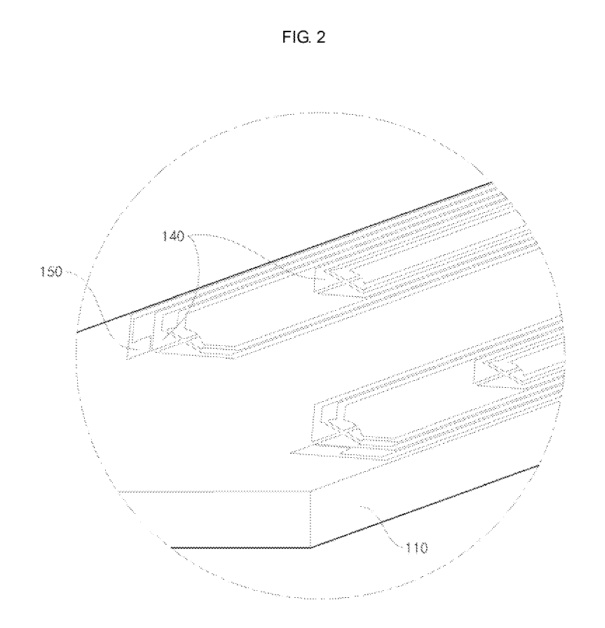

FIG. 1 is a perspective view showing a probe structure capable of measuring a pH level according to an embodiment of the present disclosure, and FIG. 2 is an enlarged view showing the A portion of FIG. 1.

Referring to FIGS. 1 and 2, a probe structure 10 of the present disclosure includes a probe unit 110, a fixing body 120, an electrode array 140, a reference electrode 150, an electric wire 160 and measured signal collecting electrodes 170a, 170b.

The probe unit 110 is inserted into a body of an experiment target such as a mouse, and its front end is processed sharp to be easily inserted into the body of the experiment target. A rear end of the probe unit 110 is fixed to the fixing body 120.

An electrode array 140 capable of collecting neural signals from the experiment target and an electric wire 160 electrically connected to the electrode array 140 are integrated at the probe unit 110.

The electrode array 140 is disposed at the front end of the probe unit 110 and senses a neural signal from the experiment target and a pH level. The electrode array 140 may include a working electrode for applying an electric stimulation to the experiment target and a neural signal recording electrode for obtaining a neural signal and a pH level signal of the experiment target changed by the working electrode. FIG. 2 shows a state where a plurality of working electrodes and a plurality of neural signal recording electrodes are formed. For forming the working electrode, IrOₓ may be deposited on the probe unit 110 by means of sputtering, and all kinds of conventional techniques may also be applied to form the working electrode.

The reference electrode 150 is disposed at the front end of the probe unit 110 to be spaced apart from the electrode array 140 by a predetermined interval and senses a reference signal for pH level measurement. Specifically, the reference electrode 150 may be made of material whose charge capacity does not easily change in order to provide the reference signal for pH level measurement even though the neural signal changes. For this, the reference electrode 150 may be made of, for example, a nanoporous Au electrode.

If comparing a pH level of the experiment target measured at the electrode array 140 and the reference electrode 150 in an initial state before an electric or optical stimulation signal is applied to the experiment target with a pH level of the experiment target measured at the electrode array 140 and the reference electrode 150 in a state where an electric or optical stimulation signal is applied to the experiment target, the pH level of the experiment target at the initial stage and the pH level of the experiment target after the change of nerve may be extracted.

The electric wire 160 is electrically connected to the measured signal collecting electrodes 170a, 170b integrated at the fixing body 120. The measured signal collecting electrodes 170a, 170b integrated at the fixing body 120 is electrically connected to a wiring of a printed circuit board (PCB) (not shown) to which the fixing body 120 is attached.

In the configuration above, a neural signal from the experiment target is collected through the electrode array 140 of the probe unit 110, and the collected neural signal may be transmitted to the outside through the electric wire 160.

The measured signal collecting electrodes may be classified into a measured signal collecting electrode 170a to which a signal sensed by the reference electrode 150 is transmitted and a measured signal collecting electrode 170b to which a signal sensed by the electrode array 140 is transmitted.

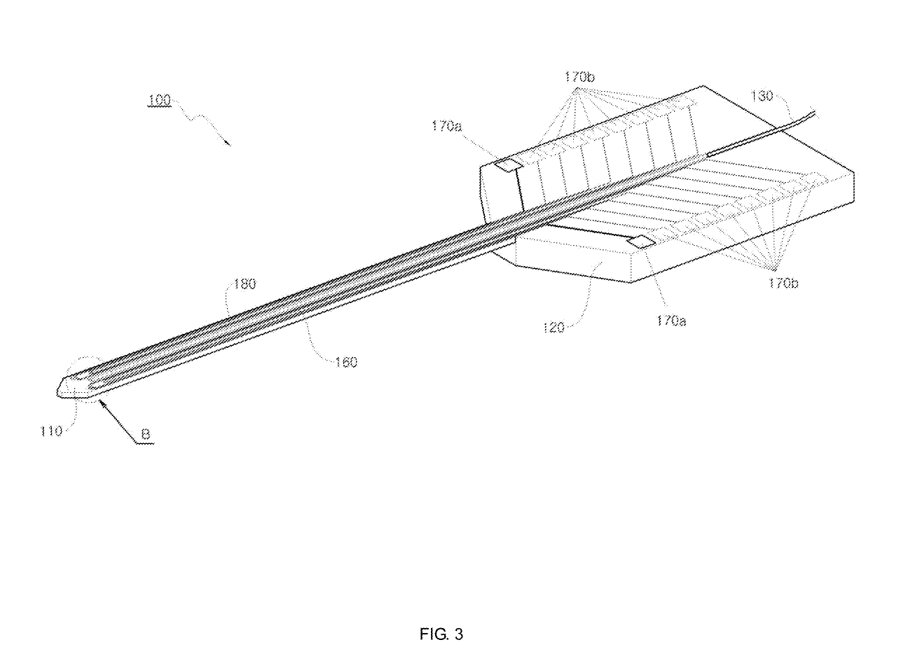

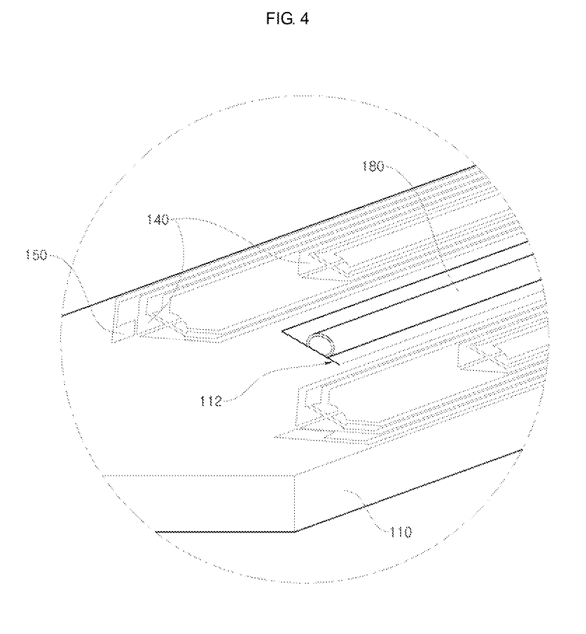

FIG. 3 is a perspective view showing a probe structure capable of measuring a pH level according to another embodiment of the present disclosure, and FIG. 4 is an enlarged view showing the B portion of FIG. 3.

Referring to FIGS. 3 and 4, a probe structure 100 of the present disclosure further includes an optical stimulation structure for stimulating a nerve of an experiment target by light in addition to electric stimulation by a working electrode.

For this, an optical fiber 130 is connected to the fixing body 120, and an exterior optical stimulation may be transmitted through the optical fiber 130. In order to transmit an optical signal input from the optical fiber 130 to the probe unit 110, an optical waveguide 180 may be fixed and attached to the surface of the probe unit 110. In addition, a groove 112 may also be formed in the probe unit 110 along the longitudinal direction of the probe unit 110 to accommodate the optical waveguide 180.

According to another embodiment of the present disclosure, in order to transmit the optical signal input from the optical fiber 130, the probe unit 110 may be made of material capable of transmitting an optical signal without having the optical waveguide 180. In detail, the probe unit 110 may be made of glass which is light transmission material. Since glass is strong against heat, even though an optical signal of a high power is irradiated, the shape of the glass does not deform. In addition, due to a low light loss factor, the loss of light may be greatly decreased while optical signals are transmitted through the probe unit 110.

In addition, the probe unit 110 may also be made of polymer which is light transmission material. Polymer allowing light transmission may use PMMA, PS, PPDMS, SU-8, COC or the like, without being limited thereto.

Moreover, instead of using the optical fiber 130 which transmits optical signals from the outside, a light source such as an LED may be directly fixed to the fixing body 120 to transmit optical signals to the probe unit 110. In other words, if an optical stimulation signal may be transmitted to the probe unit 110, a light source for directly generating light as well as a member for transmitting an optical simulation signal of an external light source may be applied to the present disclosure.

The probe structure 10, 100 of the above embodiments allows a neural signal and a pH level to be analyzed by stimulating nerves of an experiment target and collecting resultant neural signals and pH level measured signals.

For this, first, the probe unit 110 is inserted into a body portion of the experiment target, for example the brain. In a state where the probe unit 110 is inserted, an electric stimulation signal or an optical stimulation signal is applied to the body portion by using the working electrode or the optical waveguide 180.

The nerves of the experiment target generate neural signals as a response to the applied electric stimulation or optical stimulation, and the generated neural signal and changed pH level are sensed by the electrode array 140 and the reference electrode 150. The neural signal information and the pH level measurement information sensed by the electrode array 140 and the reference electrode 150 are transmitted to the measured signal collecting electrodes 170a, 170b through the electric wire 160. Subsequently, the information is transmitted to an external computer through PCB or the like connected to the measured signal collecting electrodes 170a, 170b, and the external computer may analyze nerve activities and the change of a pH level of the experiment target by using the information.

Since the probe structure 10, 100 of the present disclosure additionally includes the reference electrode 150 with a simple and thin film structure, the pH level change information may be collected together with the neural signal information without changing size and thickness of the probe structure. Therefore, compared with a conventional method where a probe structure is applied to measure neural signals and a pH sensor is separately applied to measure a pH level, the probe structure of the present disclosure may obtain precise measured signals with a simple procedure.

While the exemplary embodiments have been shown and described, it will be understood by those skilled in the art that various changes in form and details may be made thereto without departing from the spirit and scope of the present disclosure as defined by the appended claims.

What is claimed is:

1. A probe structure capable of measuring a pH level, comprising:
a probe unit inserted into an experiment target;
a fixing body connected to a terminal of the probe unit and fixing the probe unit;
an electrode array disposed at a front end of the probe unit and sensing a neural signal from the experiment target and a pH level;
a reference electrode disposed at the front end of the probe unit to be spaced apart from the electrode array by a predetermined interval and sensing a reference signal for pH level measurement;
an electric wire electrically connected to the electrode array and the reference electrode; and
a measured signal collecting electrode integrated at the fixing body and collecting measured signals transmitted through the electric wire.

2. The probe structure capable of measuring a pH level according to claim 1, wherein the electrode array includes:
a working electrode for applying an electric stimulation to the experiment target; and
a neural signal recording electrode for obtaining a neural signal of the experiment target changed by the working electrode.

3. The probe structure capable of measuring a pH level according to claim 1, further comprising an optical fiber for transmitting an optical signal to the probe unit.

4. The probe structure capable of measuring a pH level according to claim 3, wherein the probe unit is made of glass which allows optical signal transmission.

5. The probe structure capable of measuring a pH level according to claim 3, wherein the probe unit is made of polymer which allows optical signal transmission.

6. The probe structure capable of measuring a pH level according to claim 3, further comprising an optical waveguide connected to the optical fiber and fixed and attached to the surface of the probe unit along the longitudinal direction of the probe unit.

7. The probe structure capable of measuring a pH level according to claim 6, wherein a groove is formed in the probe unit along the longitudinal direction to accommodate the optical waveguide.

8. The probe structure capable of measuring a pH level according to claim 1, wherein the reference electrode is made of a nanoporous Au electrode to minimize the change of charge capacity.
